(19) United States
(12) Patent Application Publication
(10) Pub. No.: US 2023/0201046 A1
(43) Pub. Date: Jun. 29, 2023

(54) METHOD FOR PRODUCING A NONWOVEN ELEMENT FOR HYGIENE ARTICLES

(71) Applicant: Nitto Advanced Nonwoven Ascania GmbH, Aschersleben (DE)

(72) Inventors: Jan Michael TRINKAUS, Euskirchen (DE); Raik HUEPFEL, Arnstein (DE)

(73) Assignee: Nitto Advanced Nonwoven Ascania GmbH, Aschersleben (DE)

(21) Appl. No.: 18/087,906

(22) Filed: Dec. 23, 2022

(30) Foreign Application Priority Data
Dec. 27, 2021 (DE) 10 2021 006 353.8

(51) Int. Cl. A61F 13/15; A61K 8/02; D04H 5/03; D04H 13/00; D04H 3/16; D04H 1/4291

(52) U.S. Cl. CPC A61F 13/15577; A61K 8/0208; D04H 1/4291; D04H 3/16; D04H 5/03; D04H 13/001; A61F 2013/15934

(57) ABSTRACT

A method for producing a nonwoven element for hygiene articles is accomplished by the steps of: forming a fibrous web from a multi-ply nonwoven material with at least one carded staple fiber layer and a storage layer which is arranged on the staple fiber layer and which has cellulose fibers, wherein at least a portion of the staple fibers of the staple fiber layer are formed from a thermoplastic; applying liquid jets to the fibrous web, as a result of which the fibers of the multi-ply nonwoven material are intermingled and entangled, and the fibrous web is embossed with a surface structure; applying heat to the fibrous web, as a result of which the thermoplastic staple fibers at least partially fuse and the fibrous web is bonded to form a nonwoven web, and severing individual nonwoven elements from the nonwoven web.

### CROSS-REFERENCE TO RELATED APPLICATIONS

This application claims priority under 35 USC 119 of German Application No. DE 10 2021 006 353.8, filed on Dec. 27, 2021, the disclosure of which is herein incorporated by reference.

### BACKGROUND OF THE INVENTION

The invention is directed to a method for producing a nonwoven element for hygiene articles, particularly for sanitary wipes.

Sanitary wipes are routinely used for cleaning of surfaces, whether surfaces of objects or body surfaces. A typical case of application, for example, is the cleaning and disinfection of hands, in which case the sanitary wipes are usually used in a damp state in which the corresponding sanitary wipe has been soaked in a cleaning agent and/or disinfecting agent previously and, in particular, in the course of production. Alternatively, sanitary wipes can also serve as conventional cleaning towels for absorbing liquids and picking up dust or dirt, in which case it is also possible to use them in a dry state. The sanitary wipes are also used as a component of cleaning devices. This includes, for example, use in a robotic vacuum cleaner or with simple handles to which the corresponding sanitary wipes are fitted.

When damp sanitary wipes are used, the need to keep the cleaning liquid inside the sanitary wipe and to dispense the cleaning liquid when needed or when exerting pressure against the surface is a particular priority. At the same time, the sanitary wipe must be so constituted that the dirt removed by the cleaning liquid is picked up by the sanitary wipe in a suitable manner. A relatively large specific surface is needed for this purpose, which is usually achieved by means of the utilized materials on the one hand and by an embossed surface structure on the other hand.

With this in mind, a layer of cellulose, also referred to as pulp layer, is usually provided. This cellulose layer is suitable for absorbing relatively large amounts of liquid. In the case of damp sanitary wipes, for example, this liquid can be the cleaning liquid. But even when using dry cleaning towels, a layer of this kind can be useful for suitably removing liquid from surfaces.

This cellulose layer is arranged on and fastened to a further layer which is usually a layer of nonwoven material, since this material can be produced inexpensively on the one hand and has a sufficient cleaning capacity and stability on the other hand. Nonwoven materials are also commonly referred to as formed fabric and differ from knitted or woven materials in that individual fibers are aligned substantially loosely. This alignment of individual fibers is also referred to as a fibrous web. This fibrous web must be bonded in a subsequent process step in order to develop a sufficient strength and stability. This bonding can be carried out mechanically, thermally or chemically, for example, the fibers being connected or entangled in this way.

Beyond this, a distinction is also made in nonwoven materials between the types of fibers used. For example, one possibility consists in combining continuous filaments to form a so-called spunbond. These continuous filaments are usually extruded together, which already results to a certain extent in an arrangement in production direction. However, exclusively thermoplastic fiber materials are usable for forming nonwovens in this manner.

Alternatively, it is also possible to consolidate individual short fibers - also known as staple fibers - to form a staple web. To this end, the individual staple fibers are usually oriented in production direction in a carding process.

On the whole, spunbonds are distinguished by a substantially higher stiffness and strength owing to the long fiber lengths, while staple webs form a soft, more voluminous surface because of the short fiber lengths. Further, staple webs can also be formed from materials which are not thermoplastic and therefore not extrudable. Accordingly, staple webs are usable in a very versatile manner.

Methods for producing nonwoven elements for sanitary wipes are known in general from the prior art. For example, EP 1 775 116 B1 describes a multi-ply nonwoven element with two outer fiber layers which can be formed as carded staple fiber layers or as spun fiber layers and which enclose a layer of cellulose. This multi-ply construction is then consolidated by applying water jets, as a result of which the fibers of the outer layer are bonded in particular and thus ensure a sufficient strength.

EP 2 010 703 B1 also discloses a multi-ply construction having two fibrous web layers made from formed fabric which enclose a layer of cellulose. This construction is then also consolidated by applying water jets and by means of air through bonding (ATB). In air through bonding, thermoplastic materials which fuse under the action of heat and bond with one another in this way are used in particular in the outer layers. In addition to the entanglement of the individual fibers which is achieved by applying water jets, there is an additional consolidation in which the individual fibers are bonded together.

The methods used up to this point have generally proven successful. However, there is an increasing demand for hygiene articles which are usable in a particularly flexible manner for different fields of application and which, further, are characterized by a highly effective cleaning action and good stability.

### SUMMARY OF THE INVENTION

With this in mind, the invention teaches a method for producing a nonwoven element for hygiene articles according to claim 1, a nonwoven element for hygiene articles according to claim 15 and a hygiene article according to claim 19.

The method according to the invention provides that a fibrous web is formed from a multi-ply nonwoven material with at least one carded staple fiber layer and a storage layer arranged on the staple fiber layer, the storage layer having at least cellulose fibers and the at least one staple fiber layer having at least partially thermoplastic, therefore, fusible staple fibers. Subsequently, the fibrous web is preferably acted upon on both sides by liquid jets, as a result of which the fibers of the multi-ply nonwoven material are intermingled and entangled, and a surface structure is embossed into the fibrous web at the same time.

As a result of the embossing, a three-dimensional structure characterized by highly compressed areas and less compressed or non-compressed areas is imparted to the nonwoven web. Correspondingly, the nonwoven web has areas with a higher surface density and areas with a lower surface density. Since the staple fibers are also mechanically hooked together more firmly in the more compressed areas, these areas are also characterized by increased strength. This effect is further heightened by the subsequent at least partial fusing of the staple fibers. Therefore, the embossing is essential for stabilizing the nonwoven web.

Beyond this, the embossing also enlarges the surface of the nonwoven material and increases roughness. In addition to punctiform and grid-shaped impressions, the nonwoven material can also be provided in particular with line-shaped or wave-shaped impressions.

The fibrous web is then acted upon by heat, as a result of which the thermoplastic staple fibers at least partially fuse and, after a subsequent cooling, the fibrous web is consolidated to form a nonwoven web. It is self-evident within the framework of the invention that the nonwoven web already forms a nonwoven element according to the invention and, finally, individual cut nonwoven elements can be separated from the nonwoven web. This application of heat can preferably be carried out over the course of an air through bonding. In this case, the nonwoven web moves through a heating oven where it is acted upon by hot air. A heating oven of this type is usually provided during the production of nonwovens in order to bring about a bonding of the individual fibers.

Accordingly, it is provided that the nonwoven element produced by means of the method according to the invention has at least one carded staple fiber layer so that a particularly soft surface results and a very flexible use of different fiber materials is possible at the same time. However, when using staple fibers a consolidation is required at the same time because the tensile strength is substantially lower compared to continuous filaments. This is achieved on the one hand by means of the two-step consolidation with liquid jets by means of which the staple fibers of the staple fiber layer and of the storage layer are entangled with one another and on the other hand by means of the application of heat, as a result of which staple fibers which adjoin one another undergo a bonding connection.

The storage layer preferably has not only cellulose fibers but also staple fibers, in particular staple fibers of a thermoplastic material. By incorporating staple fibers made of thermoplastic material, the storage layer can be connected to a considerable extent to the at least one staple fiber layer. This results in a particularly good stabilization of the storage layer and connection to the staple fiber layer.

The staple fibers of thermoplastic material are preferably selected and adapted to the staple fibers used in the staple fiber layers so that, in the course of fusing, the fused thermoplastic material can penetrate in each instance into the adjoining layers and accordingly ensure a sufficient connection. The cellulose fibers in the storage layer preferably have a length of between 4 and 10 mm, particularly preferably between 6 and 8 mm.

According to a preferred further development, it is provided that the multi-ply nonwoven material has a further fiber layer, the storage layer is arranged between the staple fiber layer and the further fiber layer, the further fiber layer also being at least partially formed from thermoplastic fibers. In this way, the storage layer is encapsulated between two outer fiber layers, and a sufficiently strong bond can be provided at the same time through the use of thermoplastic fibers in the two outer fiber layers and in the storage layer. The two outer fiber layers can be formed identically in principle. However, an embodiment in which the two outer fiber layers differ from one another with respect to their basis weight and/or the utilized fibers and/or with respect to the utilized fiber materials is preferable. The individual surfaces of the nonwoven elements can be formed for different application purposes in this way.

For example, one possibility consists in that a further fiber layer is formed as a spun fiber layer comprising continuous filaments such that the one side of the nonwoven element is formed to a sufficient extent for stability and tensile strength and the other side for a particularly soft quality. In this way, the characteristics of the different kinds of formed fabrics are advantageously combined. Alternatively, however, both outer fiber layers can also be formed as carded staple fiber layer so that the flexibility with respect to the use of different fiber materials is substantially increased.

Regardless of the specific configuration of the two fibrous web layers, the production of the fibrous web is carried out in a multi-step process, a first fiber layer being formed initially either in a spunbonding method or by carding staple fibers. The storage layer is then arranged on this first fiber layer in a so-called air-laid process, wherein individual staple fibers and cellulose fibers are loosely laid on the first fiber layer. In this context, the term “cellulose fibers” means fibers comprising a cellulose material, e.g., paper, which are formed substantially shorter than the staple fibers. In contrast to a process in a carding machine, the fibers of the textile layer are accordingly not oriented in production direction but rather only loosely laid on. A further fiber layer can then be arranged on the textile layer. In case a first fiber layer comprises spunbond, it is compulsory that this further fiber layer is a fiber layer of carded staple fibers which is supplied from a carding machine. Insofar as the first fiber layer is already a staple fiber layer, this production step can be dispensed with in principle within the framework of the invention. However, it is also then preferable if the storage layer is arranged between two carded staple fiber layers.

According to a preferred further development of the invention, the thermoplastic staple fibers in the at least one staple fiber layer are formed from a thermoplastic material and/or as bicomponent fibers having a core and a cladding of thermoplastic material. In contrast, the core is formed from a non-thermoplastic material or from a thermoplastic material which has a higher melting temperature than the thermoplastic material of the cladding. The use of bicomponent fibers is particularly advantageous in this connection because, on the one hand, the extent to which a fusing or a bonding connection between individual staple fibers is advisable can be determined to a sufficient extent, while the core retains its structure at the same time and a sufficient stability is also ensured after fusing. In principle, the thermoplastic staple fibers can be formed in their entirety as bicomponent fibers.

Further, the staple fibers can also be formed with non-round cross sections (shaped fibers). Because of the non-round cross section, the surface of the fibers is increased with the basis weight of the formed fabric and the weight per length of the fibers remaining the same. The visual appearance can also be particularly advantageous.

In order to ensure that the staple fiber layers are sufficiently consolidated, it is provided that the proportion of thermoplastic staple fibers in one of the staple fiber layers, respectively, amounts to at least 10 wt%, preferably at least 15 wt%, particularly preferably at least 20 wt%. When a spun fiber layer is incorporated, the continuous filaments utilized therein are formed exclusively from a thermoplastic material in any case.

When using bicomponent fibers in the at least one staple fiber layer, it is preferably provided that the core is formed from a material having a change in length, particularly shrinkage, of less than 10% at a temperature of 170° C. This ensures that the core also remains sufficiently stable when acted upon by heat and that the structure of the nonwoven material is not altered excessively. Alternatively, it can also be explicitly provided that the fibers undergo a certain shrinkage in order to increase the abrasive effect.

At the same time, the thermoplastic material of the staple fibers in the at least one staple fiber layer and/or in the storage layer has a melting temperature of less than 170° C. Alternatively, the thermoplastic material in the storage layer can also have a lower melting temperature than in the staple fiber layers. In particular, the temperature difference can amount to between 10 and 50 K, preferably between 20 and 40 K.

Polyolefins, particularly those selected from the group comprising polyethylene (PE) or polypropylene (PP), or thermoplastics comprising renewable feedstocks, in particular selected from the group comprising polylactic acid (PLA) and polyhydroxylalkanoates (PHA), are particularly contemplated for the thermoplastic material in the staple fiber layers and/or in the storage layer. For example, all thermoplastic materials can have the same thermoplastic material. In order to reduce production costs generally, the continuous filaments of the spun fiber layer are also preferably formed from the above-mentioned materials. In particular, a polyolefin, preferably polypropylene, is particularly suited. When using bicomponent fibers, the core may also be formed from polylactic acid (PLA) or polyhydroxylalkanoates (PHA) because these materials are obtainable with different thermal properties. For example, the core can be formed from PLA1 and the cladding can be formed from PLA2, where PLA1 has a higher melting point than PLA2.

Due to the fact that at least one staple fiber layer of carded staple fibers is provided, staple fibers which are not formed from a thermoplastic material can also be used in this fiber layer. Such staple fibers can be viscose or natural fibers, for example. In particular, the material of the natural fibers is selected from the group comprising cotton, wool, linen, hemp, cellulose and flax. By using natural fibers, the nonwoven elements formed therefrom can be produced in a particularly sustainable manner. Beyond this, the nonwoven elements can also be biologically recycled in a favorable manner. A further increase in sustainability can be achieved in that the fibers are at least partially formed from recycled materials.

The method according to the invention can also provide that the nonwoven web is thermally smoothed on one side. This has to do with a method in which the nonwoven web is guided on only one side along a heated surface and the nonwoven web is pressed to a certain extent on the heated surface. This method may be compared to an ironing process as a result of which the nonwoven web is additionally consolidated on one side and various bonding steps can accordingly be initiated on both sides of the nonwoven web. Further, the thermoplastic staple fibers are additionally fused to a certain extent and the one side of the nonwoven web is accordingly smoothed.

Beyond this, the method according to the invention can be supplemented in that the nonwoven material is sprayed with additional chemical liquids or soaked in these chemical liquids. For example, the chemical liquid can be a saline solution which reduces the absorption of cationic disinfectants. Further, the use of binders which substantially reduce lint formation is also possible. Lastly, surface-active substances can also be used. These allow a configuration of the nonwoven element which is initially in a dry condition, and a liquid in the storage layer only passes outward when used. Hygiene articles which are formed from such material are commonly referred to as “dry-until-use wipes”.

A further subject matter of the invention is a nonwoven element for hygiene articles which is obtainable particularly by means of the method according to the invention. This nonwoven element has a multi-ply nonwoven material which is embossed with a surface structure and which has at least one carded staple fiber layer and a storage layer which is arranged on the staple fiber layer and which comprises staple fibers and cellulose fibers, and the fibers of the nonwoven material are entangled with one another and at least partially bonded with one another. The bonding connection can be achieved by at least partially fusing the fibers together.

Basically, all of the material features mentioned in relation to the method according to the invention and in particular the configuration of the nonwoven material also apply to this nonwoven element. This includes particularly the arrangement of the individual fiber layers or fiber plies and the utilized materials.

In particular, it is provided that the storage layer is also arranged between two outer fiber layers in the nonwoven element. These are either carded staple fiber layers in both cases or one of the fiber layers is formed as a spun fiber layer comprising continuous filaments.

It is preferably provided that at least one of the fiber layers has fibers comprising a lipophilic and/or hydrophilic material. Fibers with a coating of a lipophilic and/or hydrophilic material are also contemplated. By “hydrophilic materials” is meant materials which are soluble in water. In contrast, lipophilic materials are soluble in fat. As a result of these properties, both fatty and non-fatty dirt particles can easily be picked up in particular.

The nonwoven element is further distinguished by a basis weight of between 40 and 150 g/m². The storage layer has a basis weight of between 10 and 70 g/m², preferably between 20 and 50 g/m², while the two outer fiber layers, respectively, have a basis weight of between 5 and 70 g/m², preferably between 10 and 50 g/m².

A further subject matter of the invention is a hygiene article, particularly a sanitary wipe, with or comprising a nonwoven element according to the invention.

This nonwoven element can preferably be at least partially impregnated with a cleaning liquid.

Further, the nonwoven element can be provided with a structure, particularly an embossed structure, on one side. The nonwoven element then has areas with a greater hardness or compression resulting in additional stability. At the same time, the surface has a greater roughness, as a result of which the cleaning performance can be improved. Accordingly, the structured side preferably forms the cleaning side.

Further, connection elements are preferably provided for mechanically fastening to a cleaning unit. This cleaning unit can be, for example, a robotic vacuum cleaner, a vacuum cleaner, a handle or the like.

### BRIEF DESCRIPTION OF THE DRAWINGS

The invention will be described in more detail in the following referring to the initial example. In the drawings:

FIGS. 1 and 2 show methods according to the invention for producing a nonwoven element;

FIG. 3 shows a nonwoven element produced by the method according to FIG. 1;

FIG. 4 shows a nonwoven element produced by the method according to FIG. 2; and

FIG. 5 shows the nonwoven element according to FIG. 3.

### DETAILED DESCRIPTION OF THE EMBODIMENTS

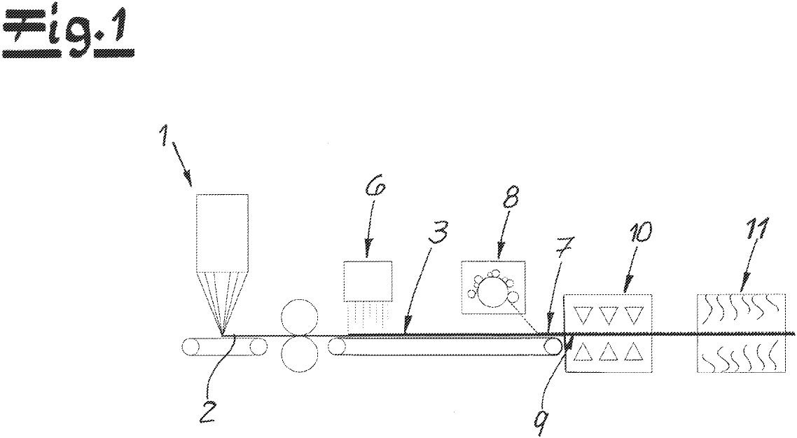

FIG. 1 shows a method according to the invention, a first fiber layer 2 being formed as spun fiber layer by means of a spun fiber extruder 1. Accordingly, the first fiber layer 2 has a multiplicity of continuous filaments 20. The continuous filaments 20 are formed from polypropylene. The spun fiber layer is formed generally with a basis weight of between 10 and 18 g/m².

In a further process step, storage layer 3 comprising staple fibers 4 and cellulose fibers 5 is laid on the first fiber layer 2. This is carried out in a so-called air-laid process with an air-laid machine 6 which loosely lays the staple fibers 4 and the cellulose fibers 5 of the storage layer 3 on the first fiber layer 2.

There follows the arrangement of a second fiber layer 7 which is formed as a carded staple fiber layer with a multiplicity of staple fibers and which is arranged in such a way that the storage layer 3 is arranged between the first fiber layer 2 and the second fiber layer 7. In this connection, the individual staple fibers of the second fiber layer 7 are aligned via a carding machine 8 and supplied as a cohesive fiber layer 7.

Accordingly, a multi-ply nonwoven material is formed from two fiber layers 2, 7 and a storage layer 3 which is arranged between the fiber layers 2, 7. At least the second fiber layer 7, as staple fiber layer, and also the storage layer has thermoplastic staple fibers 4 which are correspondingly formed at least partially from a thermoplastic material.

In a subsequent process step, the fibrous web 9 constructed in this way from a multi-ply nonwoven material is acted upon by liquid jets in an apparatus 10 in such a way that the fibers 10 of the fibrous web 9 are intermingled and entangled. At the same time, the fibrous web 9 is embossed with a surface structure. The layers of multi-ply nonwoven material which initially lie on one another relatively loosely are interconnected by mechanically entangling the fibers and the fibrous web 9 is accordingly consolidated.

In a subsequent step, the nonwoven material of the fibrous web 9 which is consolidated in this way can be fed to an oven 11 so that the fibrous web 9 is acted upon by hot air or by heat. In this respect, it is essential that the continuous filaments of the first fiber layer 2 and the thermoplastic staple fibers 4 of the second fiber layer 7 and of the storage layer 3 are at least partially formed from a thermoplastic material. Under the influence of heat, the fibers fuse and bond to one another. In this way, the storage layer 3 in particular is stabilized between the first fiber layer 2 and the second fiber layer 7, and the multi-ply structure of the fibrous web 9 is secured. The oven 11 is operated for this purpose at a temperature of at least 170° C., the temperature being governed by the melting temperature of the different fiber materials.

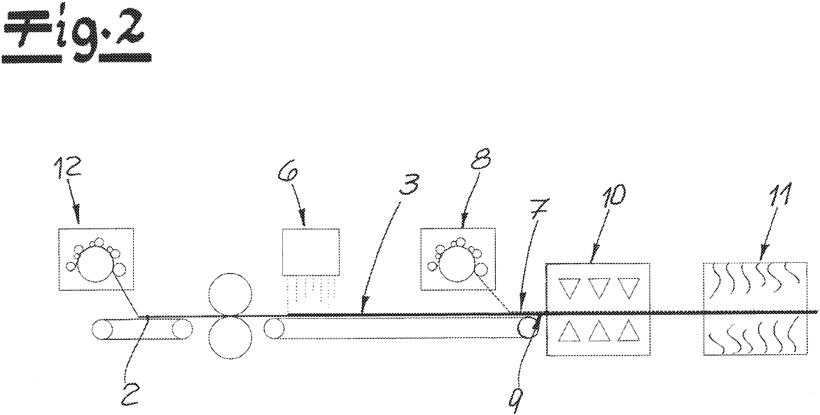

FIG. 2 shows an alternative method in which a further, second carding machine 12 which forms the first fiber layer 2 as well as the staple fiber layer is provided instead of a spun fiber extruder 1. The further process steps correspond to those in FIG. 1.

Accordingly, the nonwoven webs 9 differ in that exclusively staple fiber layers are provided according to FIG. 2, while a fiber layer is formed as spun fiber layer according to FIG. 1.

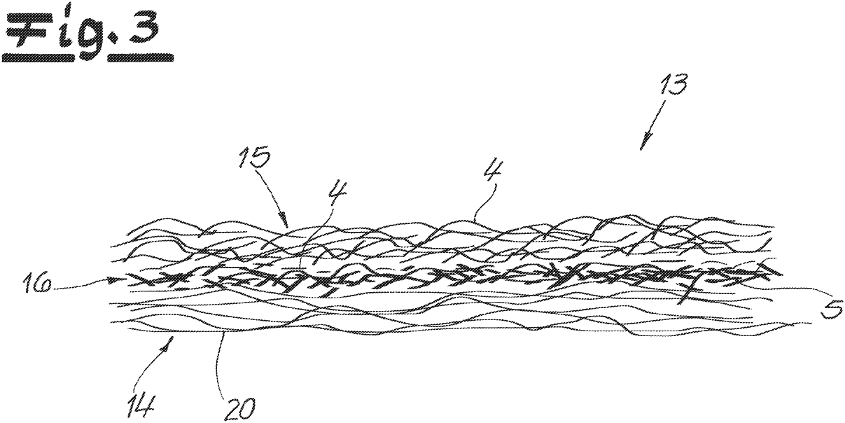

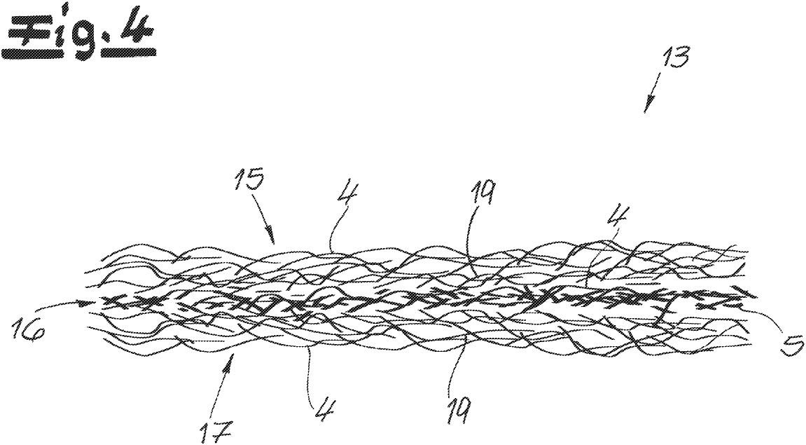

FIG. 3 shows a nonwoven element 13 which was formed with a method according to FIG. 1 by cutting out from the nonwoven web 9. The individual bonding steps are shown neither in FIG. 3 nor in FIG. 4. The nonwoven element 13 is accordingly distinguished by a three-ply construction with spun fiber layer 14 and a staple fiber layer 15 which is formed from carded staple fibers. A storage layer 16 which is formed from thermoplastic staple fibers 4 and cellulose fibers 5 is arranged between the staple fiber layer 15 and spun fiber layer 14. According to FIG. 4, two staple fiber layers 15, 17 are provided proceeding from the staple fiber layers according to FIG. 2. In both cases, thermoplastic staple fibers 4 which are formed as bicomponent fibers with a core of a non-thermoplastic material and a cladding formed from a thermoplastic material are provided in the staple fiber layers 15, 17. However, it is sufficient in principle when only one of the spun fiber layers 15, 17 has thermoplastic staple fibers 4. The continuous filaments 20 and the spun fiber layer 14 are also formed from a thermoplastic material.

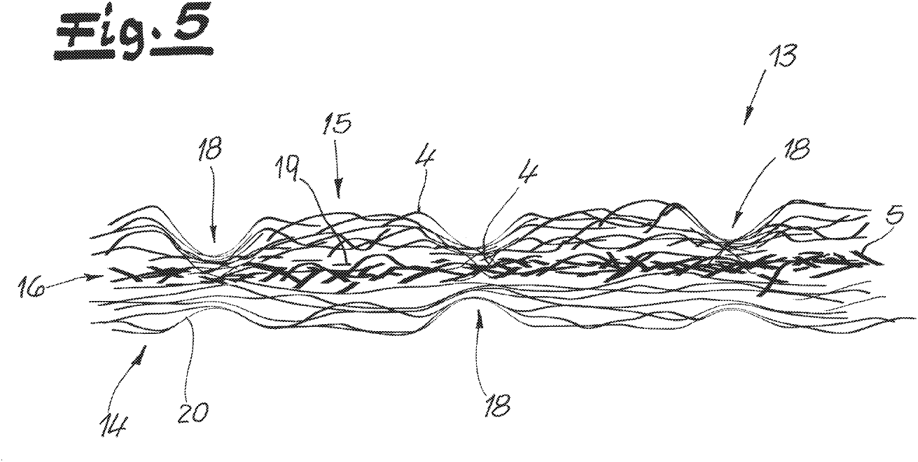

FIG. 5 shows a nonwoven element 13 according to the invention corresponding to FIG. 3 in which a surface structure in the form of indentations 18 is now visible in addition. Further, the individual fibers were also interconnected by means of liquid jets.

Further, it is provided according to the invention that the storage layer 16 is formed at least partially from non-thermoplastic staple fibers 19, particularly from natural fibers. These natural fibers can be cotton, wool, linen, hemp, flax or the like, for example.

### Example 1

In connection with a first embodiment example, a nonwoven element 13 was formed with an outer spun fiber layer 14 of polypropylene and with a basis weight of 12 g/m². The storage layer 16 has a basis weight of 28 g/m² and is enclosed by a staple fiber layer 15 of carded thermoplastic staple fibers 4 having a core of polyethylene terephthalate and a cladding of polyethylene. The staple fibers have a fineness of 1.7 dtex. The basis weight of the staple fiber layer 15 amounts to 20 g/m². The nonwoven element further has an embossed surface structure in the form of punctiform depressions.

### Example 2

According to a further embodiment example, a nonwoven element 13 was formed with an outer spun fiber layer 14 of polypropylene and a basis weight of 15 g/m². The storage layer 16 is made from Lyocell staple fibers and has a basis weight of 50 g/m². Lyocell staple fibers are regenerated fibers of cellulose. The storage layer 16 is enclosed by a staple fiber layer 15 of carded thermoplastic staple fibers 4 with a core of polyethylene terephthalate and a cladding of polyethylene. The staple fibers have a fineness of 3.4 dtex. The basis weight of the staple fiber layer 15 amounts to 50 g/m². Further, the nonwoven element 13 has an embossed surface structure in the form of punctiform depressions.

### Example 3

According to a third embodiment example, two staple fiber layers 15, 17 are provided. The one staple fiber layer 15 is formed from staple fibers 4 of polyethylene terephthalate. The staple fibers have a fineness of 1.7 dtex. The basis weight of the staple fiber layer 15 amounts to 15 g/m². The storage layer 16 corresponds to that of Example 1 with a basis weight of 30 g/m². The second staple fiber layer 17 is formed of thermoplastic staple fibers 4 having a core of polyethylene terephthalate and a cladding of polyethylene. The fineness is 2.2 dtex, and the basis weight is 15 g/m².

### Example 4

According to a fourth embodiment example, two staple fiber layers 15, 17 are provided. The one staple fiber layer 15 is formed from thermoplastic staple fibers 4 having a core of a first polylactide and a cladding comprising a second polylactide, the melting temperature of the first polylactide being 30 K higher than that of the second polylactide. The staple fibers have a fineness of 3.4 dtex. The basis weight of the staple fiber layer 15 amounts to 25 g/m². The further staple fiber layer 17 is formed almost identically, but the fineness is only 1.7 dtex. The storage layer 16 corresponds to that of Example 1 but has a basis weight of 50 g/m².

### Example 5

According to a fifth embodiment example, two staple fiber layers 15, 17 are provided. The first staple fiber layer 15 and the second staple fiber layer 17 are formed with 50% thermoplastic staple fibers 4 having a core of polyethylene terephthalate and a cladding of polyethylene and 50% thermoplastic staple fibers 4 of polyethylene terephthalate. The fineness amounts to 1.7 dtex and the basis weight amounts to 20 g/m². The storage layer 16 corresponds to that of Example 1 but with a basis weight of 30 g/m².

### Example 6

According to a sixth embodiment example, two staple fiber layers 15, 17 are provided. The first staple fiber layer 15 is formed from staple fibers of viscose with a fineness of 1.7 dtex, and the second staple fiber layer 17 is formed from staple fibers with a core of polyethylene terephthalate and a cladding of polyethylene with a fineness of 3.4 dtex. Viscose is a regenerated cellulose. The basis weight amounts to 15 g/m² for the first staple fiber layer 15 and 20 g/m² for the second staple fiber layer 17. The storage layer 16 corresponds to that of Example 1 but with a basis weight of 30 g/m².

### Example 7

According to a seventh embodiment example, two staple fiber layers 15, 17 are formed of staple fibers of viscose with a fineness of 1.7 dtex. The basis weight amounts to 20 g/m². The storage layer 16 is formed from a mixture of Tyvek fibers (HDPE) and cellulose flakes with a basis weight of 40 g/m².

In all of the examples mentioned above, the second staple fiber layer 17 or the spun fiber layer 14 forms one side of a nonwoven element 13 which forms a cleaning side of a hygiene article, e.g., of a cleaning wipe. To this end, the fibers utilized in these layers can have a greater thickness than the staple fibers of the staple fiber layer 15. As a result of the thicker fibers, the abrasive effect is increased in the course of cleaning. Different hygiene products which have been formed from the above-mentioned nonwoven elements 13, for example, are discussed in the following.

Various hygiene products can be produced from the above-mentioned nonwoven elements 13. Various exemplary sanitary wipes are discussed in the following.

### Example 8

Sanitary wipes for wet cleaning with an equal proportion of hydrophilic fibers and lipophilic fibers in order to be able to sufficiently absorb cleaning liquid on the one hand and removed fats and dirt on the other hand. Further, the sanitary wipe has areas with high hardness. This can be achieved, for example, by means of hot embossing. Beyond this, the sanitary wipe can also have connection elements for mechanical fastening to a cleaning unit.

### Example 9

The sanitary wipe from Example 8 which is formed, however, for dry cleaning and is accordingly not saturated with a cleaning liquid. In this way, liquids can be absorbed particularly well in the course of cleaning.

### Example 10

A sanitary wipe with different sides, a first side being formed chiefly by hydrophilic fibers, and the second side constitutes the actual cleaning side. The nonwoven element 13 contacts a cleaning liquid reservoir via the first side. The cleaning liquid reservoir makes it possible to transport cleaning liquid to the second side by exerting pressure via the first side. The cleaning side is also formed with areas of high hardness in this instance. These areas increase the efficiency and the effectiveness of cleaning, while the first side allows capillary transport of cleaning liquid.

In this case also, the sanitary wipe can have connection elements for mechanically fastening to a cleaning unit.

What is claimed is:

1. A method for producing a nonwoven element for hygiene articles, comprising at least the following steps:
forming a fibrous web from a multi-ply nonwoven material with at least one carded staple fiber layer and a storage layer which is arranged on the staple fiber layer and which has cellulose fibers, wherein at least a portion of the staple fibers of the staple fiber layer are formed from a thermoplastic material,
applying liquid jets to the fibrous web, as a result of which the fibers of the multi-ply nonwoven material are intermingled and entangled, and the fibrous web is embossed with a surface structure, and
applying heat to the fibrous web, as a result of which the thermoplastic staple fibers at least partially fuse and the fibrous web is bonded to form a nonwoven web.

2. The method according to claim 1, wherein the storage layer additionally has staple fibers comprising a thermoplastic material.

3. The method according to claim 1, wherein the multi-ply nonwoven material has a further fiber layer, wherein the storage layer is arranged between the staple fiber layer and the further fiber layer, wherein the further fiber layer is formed at least partially from thermoplastic staple fibers.

4. The method according to claim 3, wherein the further fiber layer is formed as carded staple fiber layer or as spunbonded layer comprising continuous filaments.

5. The method according to claim 1, wherein the thermoplastic staple fibers in the at least one staple fiber layer are each formed at least partially from a thermoplastic material or as bicomponent fibers with a core and a cladding of a thermoplastic material.

6. The method according to claim 1, wherein the proportion of thermoplastic staple fibers in the at least one staple fiber layer is at least 20 wt%.

7. The method according to claim 5, wherein the thermoplastic staple fibers in the at least one staple fiber layer are formed with the core and the cladding of thermoplastic material and wherein the core is formed from a material which has a change in length of less than 10% at a temperature of 170° C.

8. The method according to claim 2, wherein the thermoplastic material of the thermoplastic staple fibers in the at least one staple fiber layer and/or in the storage layer has a melting temperature of less than 170° C.

9. The method according to claim 2, wherein the thermoplastic material of the thermoplastic staple fibers in the at least one staple fiber layer and/or in the storage layer is a polyolefin, selected from the group consisting of polyethylene and polypropylene.

10. The method according to claim 2, wherein the thermoplastic staple fibers of the at least one staple fiber layer and in the storage layer have the same thermoplastic material.

11. The method according to claim 1, wherein a portion of the staple fibers in the at least one staple fiber layer is formed from a non-thermoplastic material.

12. The method according to claim 11, wherein the staple fibers formed from a non-thermoplastic material are natural fibers, selected from the group consisting of cotton, wool, linen, hemp, flax and cellulose.

13. The method according to claim 1, wherein the nonwoven web is thermally smoothed on one side.

14. The method according to claim 1, wherein the nonwoven web has a basis weight of between 45 and 150 g/m².

15. The method according to claim 1, wherein the cellulose fibers have a length of between 4 and 10 mm.

16. A nonwoven element for hygiene articles, obtainable by means of a method according to claim 1, comprising a multi-ply nonwoven material which is embossed with a surface structure, at least one carded staple fiber layer, and a storage layer which is arranged on the staple fiber layer and is formed of staple fibers and cellulose fibers, wherein the fibers of the nonwoven material are entangled with one another and the staple fibers are at least partially bonded to one another.

17. The nonwoven element according to claim 16, wherein the storage layer is arranged between two fiber layers.

18. The nonwoven element according to claim 17, wherein the two fiber layers are formed as carded staple fiber layers, or wherein one of the fiber layers is formed as spunbonded layer of continuous filaments.

19. The nonwoven element according to claim 16, wherein the nonwoven element has a basis weight of between 40 and 150 g/m².

20. A hygiene article formed of the nonwoven element according to claim 16.

21. The hygiene article according to claim 20, wherein the nonwoven element is saturated at least partially with a cleaning liquid.

22. The hygiene article according to claim 20, wherein connection elements are provided for mechanical fastening to a cleaning unit.
